(19) United States
(12) Patent Application Publication
(10) Pub. No.: US 2018/0220052 A1
(43) Pub. Date: Aug. 2, 2018

(54) Temporal Modulation of Fluorescence Imaging Color Channel for Improved Surgical Discrimination

(71) Applicant: KARL STORZ Imaging, Inc., Goleta, CA (US)

(72) Inventor: Russell Granneman, Goleta, CA (US)

(73) Assignee: KARL STORZ Imaging, Inc., Goleta, CA (US)

(21) Appl. No.: 15/421,126

(22) Filed: Jan. 31, 2017

(51) Int. Cl. H04N 5/225; H04N 5/232; A61B 1/04; A61B 5/00

(52) U.S. Cl. CPC H04N 5/2256; H04N 5/23229; H04N 2005/2255; A61B 5/0071; A61B 1/043

(57) ABSTRACT

Improved fluoresced imaging (FI) and other sensor data imaging processes, devices, and systems are provided to enhance display of FI images and reflected light images together. Generally an imaging scope device observes white light (WL) and FI images, and sends them to a control module which processes them for display together in an FI modality. A flashing mode causes temporal modulation of pixel intensity values of the FI image stream to improve distinguishability of features on the display. A composite image stream is produced depicting the reflected light components and the fluoresced light component detected by the image sensor assembly. Hardware designs are provided to enable real-time processing of image streams from medical scopes.

### TECHNICAL FIELD OF THE INVENTION

The invention relates generally to the field of image capture and more specifically to a medical imaging camera systems and methods which combine fluorescent imaging with visible color imaging with improved visibility of features.

### BACKGROUND OF THE INVENTION

Endoscopes and other medical scopes often use fluorescing agents or autoflorescence to better examine tissue. A fluorescing agent such as a dye may be injected or otherwise administered to tissue, and then an excitation light is directed toward the tissue. The fluorescing agent fluoresces (emits light typically at a longer wavelength than the excitation light), allowing a sensor to detect the light, which may or may not be in a wavelength visible to the human eye. The detected light is formatted to images, and examining the images can indicate the concentration of fluorescing agent in the observed tissue. Further, a phenomenon known as autoflorescence may occur in which tissue fluoresces light under certain conditions without a fluorescing agent. Such light can be detected as well. Images based on detected fluoresced light, known as “fluorescence imaging” (FI), are therefore useful in medical diagnosis, testing, and many scientific fields.

Other medical sensing schemes such as ultrasonic or optical coherence tomography also produce data represented to the user as images. It is often necessary to display visual color images of along with the FI or other sensor images in order to properly distinguish anatomical reference features and determine all desired characteristics of the tissue being investigated. The visual color images are produced by emitting light toward the tissue, and with a camera, or image sensor, taking pictures of the reflected light. Both the reflected light images and FI images can be put into image streams to show a video of the two images to the user such as a doctor using a FI endoscope.

Systems are also known which combine or overlay FI images with reflected light images of the same area to help users interpret the data in both images, such as to identify cancerous tissue. For example, U.S. Pat. No. 9,055,862 to Watanabe et al. discloses a fluorescence imaging processing device that combines a FI image with a return-light image, and processes the images with various exponential functions based on distance.

Another document, U.S. Publication No. 2011/0164127 by Stehle et al. describes a method for showing endoscope images with fluorescent light. In this case, the fluoresced light is at visible wavelengths in the RGB color space and is detected with a visible light camera. The method seeks to enhance the fluoresced light portion of the image non-linearly by processing it to enhance the variations in the fluorescent light while de-enhancing the variations in other parts of the image's RGB color space.

Another example is the D-Light P System by KARL STORZ, which performs indocyanine green (ICG) fluorescence imaging (FI) in the near infra-red (NIR) by utilizing both fluoresced and reflected light in the FI modality.

Another method for combining FI and reflected light images is found in U.S. Pat. No. 8,706,184. In this method, the visible light image is “desaturated”, that is the colors are changed to be less colorful, and in some cases the colors are completely desaturated into grey scale images. The FI image is superimposed with the desaturated image so that fluorescent features may be clearly seen relative to the more grey version of the reflected light image. All of these techniques, and others like them, suffer from distortion of colors in the reflected light image and difficulty in distinguishing FI image features when combined with the reflected light image.

Additionally, with existing systems, it is often difficult for surgeons to visibly distinguish FI tissues with low color contrast from the surrounding tissue in the visible light image. A similar problem occurs with FI tissues having very small spatial extents in the image, even where color spaces are correctly chosen.

What is needed are improved ways to process and display fluoresced light-based images or other medical images with visible color images, and techniques to emphasize and distinguish FI images displayed together with visible light images.

### SUMMARY OF THE INVENTION

It is an object of the invention to provide improved display of fluorescence imaging (FI) images or other sensor-based images, and reflected light images, through systems and methods allowing FI or other images to be combined in a manner with improved distinguishability of FI features. This has the advantage enhancing the analytical or diagnostic benefits of providing a combined image. It is another object of the invention to provide system designs, image processing circuit designs, image processing methods and digital signal processor or graphics processing unit program code, that can process a stream of images from both FI and reflected light and combine them with the improved display techniques

Improved fluoresced imaging (FI) and other sensor data imaging processes, devices, and systems are provided to enhance display of, for example, FI images and reflected light images together. Generally an imaging scope device observes reflected light and FI images, and sends them to a control module which processes them for display together in an FI modality. A flashing mode causes temporal modulation of pixel intensity values of the FI image stream to improve distinguishability of features on the display.

A composite image stream may be produced depicting the reflected light components and the fluoresced light component detected by the image sensor assembly. Hardware designs are provided to enable near real-time processing of image streams from medical scopes.

According to a first aspect of the invention, a fluorescence imaging scope system is capable of white-light (WL) and fluorescence imaging (FI) modalities. The scope system includes an optical assembly configured to direct light received from a subject scene toward an image sensor assembly. The image sensor assembly has at least three channels and including at least one image sensor, and is configured to detect reflected light components and a fluoresced light component of the light, and produce at least three WL output signals for a WL modality and at least one FI output signal depicting the fluoresced light component for an FI modality. Image forming circuitry is configured to receive the at least three WL output signals and produce a WL image stream, and receive the at least one FI output signal and produce an FI image stream. Image processing circuitry configured to, when the image processing circuitry is in a flashing mode, cause temporal modulation of pixel intensity values of the FI image stream.

In some implementations of the first aspect, the image sensor assembly is configured to produce at least three FI output signals for the FI modality, with one or more of the at least three FI output signals depicting the reflected light component. The image forming circuitry may also be configured to receive the at least three FI output signals and produce a composite image stream depicting the reflected light components and the fluoresced light component detected by the image sensor assembly, and the image processing circuitry is configured to, when the image processing circuitry is in the flashing mode, cause temporal modulation of pixel intensity values of the composite image stream that represent the at least one FI output signal depicting the fluoresced light component through signal processing.

For example, one temporal modulation implementation may include periodically setting one or more coefficients in a color correction matrix being applied to the FI image stream to zero, thereby causing the FI image stream (or downstream equivalent depicted in the composite image stream) to flash when displayed.

In some implementations of the first aspect or the above implementations, the scope system includes an optical filter configured to attenuate the reflected fluorescence excitation light and transmit the fluoresced light.

In some implementations of the first aspect, the image processing circuitry is configured to, when the image processing circuitry is in the flashing mode, temporally modulate the at least one FI output signal or the FI image stream. In some implementations of the first aspect, the image processing circuitry is configured, in the flashing mode, to alternate periodically between an off state that suppresses, of the three channels, the one or more channels providing the at least one FI output signal, and an on state that does not suppress the one or more channels. The channels may include a red channel, a green channel, and a blue channel.

In some implementations of the first aspect or the above implementations, the image processing circuitry is further configured to compress a color space of the WL image stream into a color space not containing a fluorescence display color range for the FI image stream.

In some implementations of the first aspect or the above implementations, the image processing circuitry may further be configured to receive an external user interface control input signal controlling the flashing mode. The image processing circuitry may be configured to receive a flashing rate input for adjusting a flashing rate of the flashing mode, or to assign multiple different flashing rates to respective multiple different areas of the FI image stream based on digital image processing values calculated from the respective areas.

In some implementations of the first aspect or the above implementations, color space conversion is formed in which image processing circuitry is further configured to convert a format of the WL image stream into a second data format having a second color space larger than its original color space, while preserving color space content of the first image stream, and to format the FI image stream to a color format inside the second color space and outside the first color space.

In some implementations of the first aspect or the above implementations, image processing circuitry further includes first processing circuitry for processing the WL image stream and second processing circuitry operating in parallel with the first processing circuitry for processing the FI image stream, the first and second processing circuitry both connected to image combining circuitry and being implemented as independent parallel circuits in a field programmable gate array (FPGA).

According to a second aspect of the invention, a camera control module (CCM) is provided for commutatively coupling with a fluorescent and visible light medical scope device. The CCM includes a scope connection port configured to receive at least one output signal from a scope device, the at least one output signal including detected reflected light components for a white-light (WL) modality and detected fluoresced light components for a fluorescence imaging (FI) modality from the scope device when in use. It has image forming circuitry configured to receive the at least one output signal and produce a WL image stream and a FI image stream, and image processing circuitry configured to, when the image processing circuitry is in a flashing mode, cause temporal modulation of pixel intensity values of the FI image stream through signal processing.

In some implementations of the second aspect, the CCM is configured to receive at least three FI output signals for the FI modality, one or more of the at least three FI output signals depicting the reflected light component. The image forming circuitry is configured to receive the at least three FI output signals and produce a composite image stream depicting the reflected light components and the fluoresced light component detected by the image sensor assembly, and, when the image processing circuitry is in the flashing mode, to cause temporal modulation of pixel intensity values of the composite image stream that represent fluoresced light component. The at least three channels may include a red channel, a green channel, and a blue channel.

In some implementations of the second aspect or the above implementations, the image processing circuitry is configured to, when the image processing circuitry is in the flashing mode, temporally modulate the composite image stream or the one or more of the at least three FI output signals depicting the reflected light components.

In some implementations of the second aspect or the above implementations, the image processing circuitry is further configured to compress a color space of the WL image stream to a color space not containing a fluorescence display color range for the FI image stream. In others, the WL image stream has a first color space, and the image processing circuitry is further configured to convert a format of the WL image stream into a second data format having a second color space larger than the first color space, while preserving color space content of the WL image stream, and to format the FI image stream to a color format inside the second color space and outside the first color space.

In some implementations of the second aspect or the above implementations, the image processing circuitry is further configured to assign multiple different flashing rates to respective multiple different areas of the second image stream based on digital image processing values calculated from the respective areas.

In some implementations of the second aspect or the above implementations, image processing circuitry further includes first processing circuitry for processing the WL image stream and second processing circuitry operating in parallel with the first processing circuitry for processing the FI image stream, the first and second processing circuitry both connected to image combining circuitry and being implemented as independent parallel circuits in a field programmable gate array (FPGA).

According to a third aspect, the invention may be embodied as program code for execution on digital processing devices. Such versions include one or more tangible nontransitory computer readable media storing program code executable by a digital processing system to perform the functionality similarly to the other embodiments. The program code is executable to receive at least three signals depicting reflected light components for a WL modality and produce a WL image stream therefrom, and receive FI data depicting a fluoresced light component for an FI modality and produce an FI image stream therefrom. When the digital processing system is placed in a flashing mode, temporally modulate one of the FI data and the FI image stream, thereby causing temporal modulation of the pixel intensity values of the FI image stream through signal processing.

In some implementations of the third aspect, the program code is further executable by the digital processing system to produce a composite image stream depicting the reflected light components and the fluoresced light component detected by the image sensor assembly, and when the digital processing system is in the flashing mode, temporally modulate the fluoresced light component through signal processing. The program code may be further executable by the digital processing system to receive at least three channels for the FI modality, the WL image data carried by one or more of the three channels, and to produce a composite image stream depicting the reflected light components and the fluoresced light component detected by the image sensor assembly, and when the image processing circuitry is in the flashing mode, to alternate periodically between an off state that suppresses, of the three channels, the one or more channels providing the FI data, and an on state that does not suppress the one or more channels.

In some implementations, the program code is further executable by the digital processing system to compress a color space of the WL image stream to a color space not containing a fluorescence display color range for the FI image stream. In other implementations, the WL image stream has a first color space, and wherein the program code is further executable by the digital processing system to and to convert a format of the WL image stream into a second data format having a second color space larger than the first color space, while preserving color space content of the first image stream, and to format the FI image stream to a color format inside the second color space and outside the first color space.

In some implementations of any of the above aspects or implementations, the FI modality may include providing a (substantially) constant fluorescent excitation light (e.g., a light that is continuously on with minimal intensity variations during the FI modality).

The program code may be further executable by the digital processing system to assign multiple different flashing rates to respective multiple different areas of the FI image stream based on digital image processing values calculated from the respective areas.

Implementations of the first aspect of the invention are also implementable according to the third aspect of the invention (e.g., the various configurations and functionalities of image processing and video encoding circuitry, including a color correction matrix). According to a fourth aspect, the functionality described herein for the implementations of the first aspect may be embodied as a method of process for operating an imaging scope.

Implementations of the invention may also be embodied as software or firmware, stored in a suitable medium, and executable to perform various versions of the methods herein. These and other features of the invention will be apparent from the following description of the preferred embodiments, considered along with the accompanying drawings.

### BRIEF DESCRIPTION OF THE DRAWINGS

FIG. 1 is a hardware block diagram of an example image capture device according to an example embodiment of the invention.

FIG. 2 is a block diagram showing more of the image processing circuitry of FIG. 1.

FIGS. 3A-C show example scaling signals that may be applied to scale the FI image data.

FIG. 4 is a flowchart of a fluorescent image and visible image display process according to some embodiments.

FIG. 5 shows an example color space diagram of an example version of FIG. 4.

FIG. 6 is a flowchart of another fluorescent image and visible image display process according to other example embodiments.

FIG. 7 shows a color space diagram of an example version of the process of FIG. 6.

FIG. 8 is a flowchart of another example process including image processing to recognize elements for emphasis in the display.

### DETAILED DESCRIPTION OF EXAMPLE EMBODIMENTS

The invention provides improved display of fluorescence imaging (FI) images and reflected light images, through systems and methods that allow FI images to be combined with a nominal white light image in a manner with improved distinguishability of colors and fluorescent features, especially for small fluorescent features, thereby further enhancing the analytical or diagnostic benefits of providing a combined image. Also provided are system designs, image processing circuit designs, image processing methods and digital signal processor or graphics processing unit program code that can process a stream of image data from both FI and reflected light and combine them with the improved display techniques herein.

Because digital cameras and FI sensors and related circuitry for signal capture and processing are well-known, the present description will be directed in particular to elements forming part of, or cooperating more directly with, a method and apparatus in accordance with the invention. Elements not specifically shown or described herein are selected from those known in the art. Certain aspects of the embodiments to be described are provided in software. Given the system as shown and described according to the invention in the following materials, software not specifically shown, described or suggested herein that is useful for implementation of the invention is conventional and within the ordinary skill in such arts.

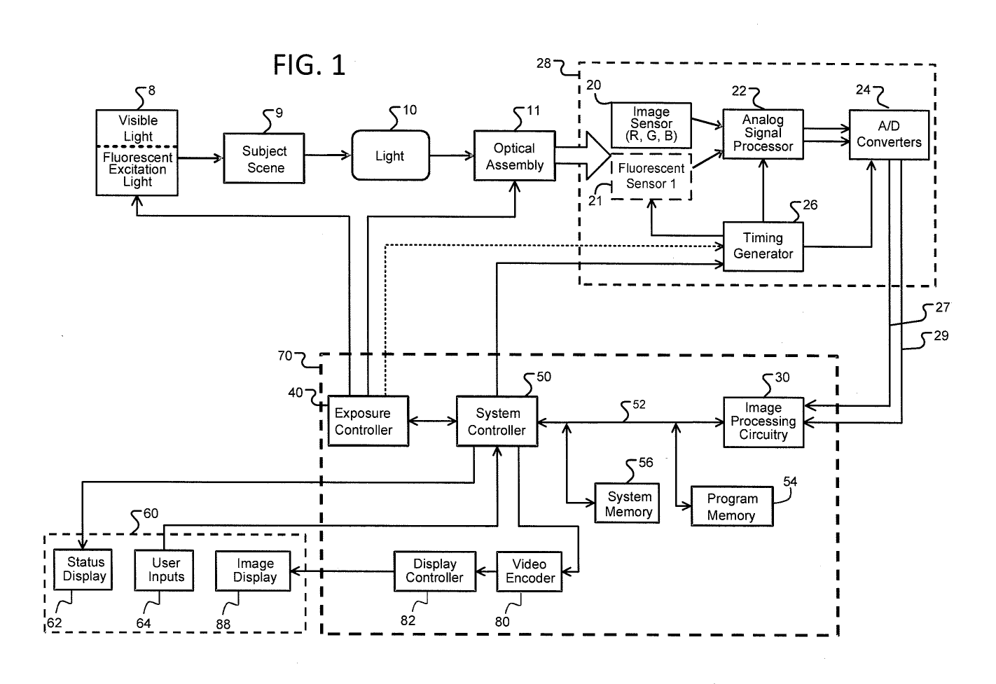

Referring to FIG. 1, a block diagram of an image capture device according to an example embodiment of the invention is shown. The invention is clearly applicable to more than one type of device enabled for image capture, such as endoscopes incorporating solid state imagers, digital microscopes, digital cameras, mobile phones equipped with imaging sub-systems, and automotive vehicles equipped with imaging sub-systems, for example. The preferred version is a fluorescence imaging scope system, such as an endoscope, capable of white-light and fluorescence imaging modalities.

A light source 8 illuminates subject scene 9 with visible light and fluorescent excitation light, which may be outside the visible spectrum in the ultra-violet range or the infra-red/near infrared range, or both. Light source 8 may include a single light emitting element configured to provide light throughout the desired spectrum, or a visible light emitting element and a one or more fluorescent excitation light emitting elements. Further, light source 8 may include fiber optics passing through the body of the scope, or other light emitting arrangements such as LEDs or laser LEDs positioned toward the front of the scope.

Any suitable known elements for emitting visible and fluorescent excitation light may be used as elements for the light emitting elements included in light source 8. In some implementations, light source 8 may be controlled so as to provide a (substantially) constant fluorescent excitation light (e.g., a light that is continuously on with minimal brightness variations) during an FI modality, but other implementations may include varying an intensity or emitted spectrum during the FI modality.

As shown in the drawing, light 10 reflected from (or, alternatively, as in the case of fluorescence, excitation light 8 absorbed and subsequently emitted by) the subject scene is input to an optical assembly 11, where the light, including both the white-light and FI components, is focused to form an image at a solid-state image sensor 20 and fluoresced light sensor 21, which are sensor arrays responsive to a designated spectrum of light.

In this version, multiple sensor arrays are employed for visible light and for fluoresced light which may include visible and invisible spectrums, but single sensor embodiments capturing both reflected and FI components are also possible. The image sensor assembly is configured to detect reflected light components and a fluoresced light component of the light, and produce at least three white-light (WL) output signals for a WL modality and at least one FI output signal depicting the fluoresced light component for an FI modality.

In some versions, a single image sensor 20 may be employed, configured as a sensor array having a spectral range of sensitivity covering visible light, near infra-red (NIR), and ultra violet light as necessary depending upon the specific application (e.g., indocyanine green (ICG) fluorescence imaging (FI)). If multiple fluorescent imaging (FI) schemes are employed, the image sensor may include a separate image sensor constructed to receive the specific wavelengths fluoresced in the various FI techniques used. While one sensor is shown, other versions may use two different fluorescent lights sensor that senses fluoresced light in the invisible ranges of IR and ultraviolet.

Optical assembly 11 includes at least one lens, which may be a wide-angle lens element such that optical assembly 11 focuses light which represents a wide field of view. Image sensor 20 (which may include separate R, G, and B sensor arrays) and fluoresced light sensor 21 convert the incident visible and invisible light to an electrical signal by integrating charge for each picture element (pixel). It is noted that fluoresced light sensor 21 is shown as an optional dotted box because many embodiments use the RGB image sensor 20 to detect the fluoresced light (e.g., NIR ICG FI). Such a scheme may be used when the fluoresced light is in a spectrum detectable by light sensor 20 that is in or near the visible light spectrum typically detected by a RGB sensor arrays.

Some such sensors have a sensitivity outside of the visible light range, such as extending slightly into the IR range or the UV range. In any case, the image sensor 20 typically produces three analog output signals that contain the visible light data of the three primary color channels, and may also contain the fluoresced light data when the fluoresced light is visible or in the range of the image sensor 20. For versions in which a separate fluorescent light sensor 21 is employed, it typically produces a single output signal. The image sensor 20 and fluoresced light sensor 21 of the preferred embodiment may be active pixel complementary metal oxide semiconductor sensor (CMOS APS) or a charge-coupled device (CCD).

The total amount of light 10 reaching the image sensor 20 and fluoresced light sensor 21 is regulated by the light source 8 intensity, the optical assembly 11 aperture, and the time for which the image sensor 20 and fluoresced light sensor 21 integrates charge. An exposure controller 40 responds to the amount of light available in the scene given the intensity and spatial distribution of digitized signals corresponding to the intensity and spatial distribution of the light focused on image sensor 20 and fluoresced light sensor 21.

Exposure controller 40 also controls the emission of fluorescent excitation light from light source 8, and may control the visible and fluorescent light emitting elements to be on at the same time, or to alternate to allow fluoresced light frames to be captured in the absence of visible light if such is required by the fluorescent imaging scheme employed. Exposure controller may also control the optical assembly 11 aperture, and indirectly, the time for which the image sensor 20 and fluoresced light sensor 21 integrate charge. The control connection from exposure controller 40 to timing generator 26 is shown as a dotted line because the control is typically indirect.

Typically exposure controller 40 has a different timing and exposure scheme for each of sensors 20 and 21. Due to the different types of sensed data, the exposure controller 40 may control the integration time of the sensors 20 and 21 by integrating sensor 20 up to the maximum allowed within a fixed 60 Hz or 50 Hz frame rate (standard frame rates for USA versus European video, respectively), while the fluoresced light sensor 21 may be controlled to vary its integration time from a small fraction of sensor 20 frame time to many multiples of sensor 20 frame time. The frame rate of sensor 20 will typically govern the synchronization process such that images frames based on sensor 21 are repeated or interpolated to synchronize in time with the 50 or 60 fps rate of sensor 20.

Analog signals from the image sensor 20 and fluoresced light sensor 21 are processed by analog signal processor 22 and applied to analog-to-digital (A/D) converter 24 for digitizing the analog sensor signals. The digitized signals each representing streams of images or image representations based on the data, are fed to image processor 30 as image signal 27, and first fluorescent light signal 29. For versions in which the image sensor 20 also functions to detect the fluoresced light, fluoresced light data is included in the image signal 27, typically in one or more of the three color channels.

Image processing circuitry 30 includes circuitry performing digital image processing functions as further described below to process and combine visible light images of image signal 27 with the fluoresced light data in signal 29. It is noted that while this version includes one fluorescent light sensor, other versions may use two different fluoresced light schemes, and some may use more than two including three, four, or more different fluoresced light imaging techniques.

Image processing circuitry 30 may provide one temporal modulation implementation by periodically setting one or more coefficients in a color correction matrix to 0, thereby causing the FI image stream (or upstream or downstream equivalent) to flash when displayed.

Timing generator 26 produces various clocking signals to select rows and pixels and synchronizes the operation of image sensor 20, analog signal processor 22, and A/D converter 24. Image sensor assembly 28 includes the image sensor 20, the analog signal processor 22, the A/D converter 24, and the timing generator 26. The functional elements of the image sensor assembly 28 can be fabricated as a single integrated circuit as is commonly done with CMOS image sensors or they can be separately-fabricated integrated circuits.

The system controller 50 controls the overall operation of the image capture device based on a software program stored in program memory 54. This memory can also be used to store user setting selections and other data to be preserved when the camera is turned off Preferably the system can be operated in a white-light (WL) modality and in a fluorescence imaging (FI) modality, either of which may be activated or deactivated by system controller 50.

Both modalities may be activated simultaneously, wherein a composite WL/FI image stream may be shown in real time as described in U.S. Publication No. US2011/0063427.

System controller 50 controls the sequence of data capture by directing exposure controller 40 to set the light source 8 intensity, the optical assembly 11 aperture, and controlling various filters in optical assembly 11 and timing that may be necessary to obtain image streams based on the visible light and fluoresced light. In some versions, optical assembly 11 includes an optical filter configured to attenuate excitation light and transmit the fluoresced light. A data bus 52 includes a pathway for address, data, and control signals.

Processed image data are continuously sent to video encoder 80 to produce a video signal. This signal is processed by display controller 82 and presented on image display 88. This display is typically a liquid crystal display backlit with light-emitting diodes (LED LCD), although other types of displays are used as well. The processed image data can also be stored in system memory 56 or other internal or external memory device.

The user interface 60, including all or any combination of image display 88, user inputs 64, and status display 62, is controlled by a combination of software programs executed on system controller 50. User inputs typically include some combination of typing keyboards, computer pointing devices, buttons, rocker switches, joysticks, rotary dials, or touch screens. The system controller 50 manages the graphical user interface (GUI) presented on one or more of the displays (e.g. on image display 88). System controller 50 may receive inputs from buttons or other external user interface controls on the scope itself (or software controls through the GUI) to receive inputs to control a flashing mode, described below, and send a control signal or command to the image processing circuitry, which is configured to receive an external user interface control input signal controlling the flashing mode.

Image processing circuitry 30 may also receive other control inputs related to the flashing mode, such as inputs to set or adjust a flashing rate or oscillation rate. For each fluoresced light signal (29) to be processed and displayed by the system, the GUI may present controls for adjusting various characteristics of temporal modulation applied to the fluoresced light images, and adjusting the transparency of the fluoresced light image when blended with the systems visible light images, as further described below. The GUI typically includes menus for making various option selections.

Image processing circuitry 30 is one of three programmable logic devices, processors, or controllers in this embodiment, in addition to a system controller 50 and the exposure controller 40. Image processing circuitry 30, controller 50, exposure controller 40, system and program memories 56 and 54, video encoder 80 and display controller 82 may be housed within camera control module (CCM) 70.

CCM 70 may be responsible for powering and controlling light source 8, image sensor assembly 28, and/or optical assembly 11. In some versions, a separate front end camera module may perform some of the image processing functions of image processing circuitry 30.

Although this distribution of imaging device functional control among multiple programmable logic devices, programmable logic devices, and controllers is typical, these programmable logic devices, processors, or controllers can be combinable in various ways without affecting the functional operation of the imaging device and the application of the invention. These programmable logic devices, processors, or controllers can comprise one or more programmable logic devices, digital signal processor devices, microcontrollers, or other digital logic circuits. Although a combination of such programmable logic devices, processors, or controllers has been described, it should be apparent that one programmable logic device, digital signal processor, microcontroller, or other digital logic circuit can be designated to perform all of the needed functions. All of these variations can perform the same function and fall within the scope of this invention.

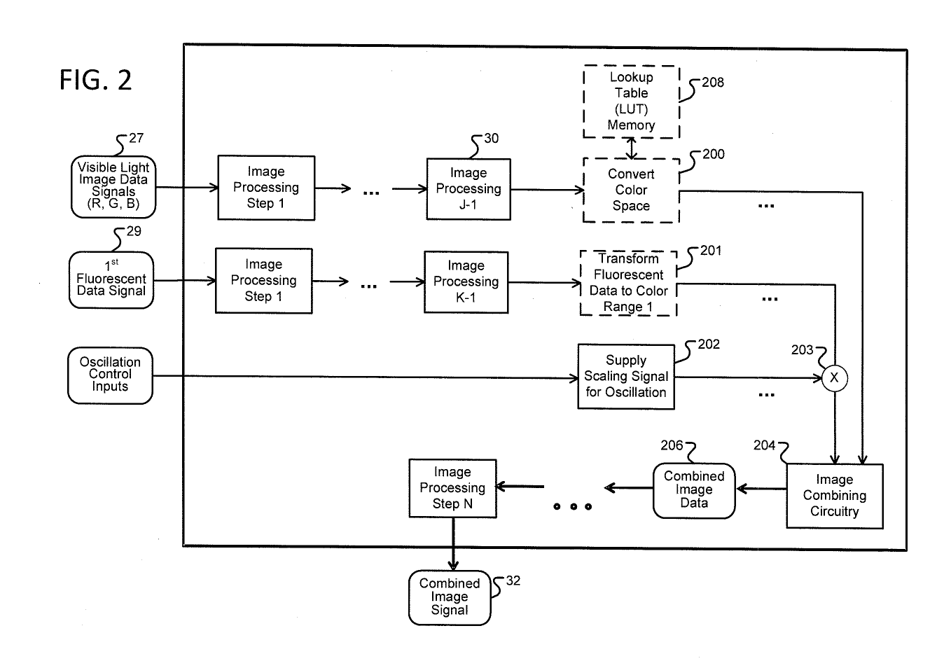

FIG. 2 shows in more detail the example image processing circuitry 30 from FIG. 1, which performs digital image processing functions for a white-light imaging modality to process and combine visible light images of image signal 27 with the fluoresced light data in signal 29 to produce the desired form of image from the data received.

Generally, A/D converter 24 or image processing circuitry 30 includes the image forming circuitry configured to receive the at least three WL output signals and produce a WL image stream, and receive the at least one FI output signal and produce an FI image stream. Image processing circuitry 30 receives the image streams and is configured to, when, for example, the image processing circuitry is placed in a flashing mode, cause temporal modulation of pixel intensity values of the FI image stream. Image signal 27 and the fluoresced light data in signal 29 are shown being fed to image processing circuitry 30 on the left side of FIG. 2.

Other versions may receive only RGB data which may include fluoresced light in one or more of the RGB channels. For image signal 27, circuitry may perform various processing steps, including converting color space at circuitry block 200. The color space conversion may involve compressing the color space to allow better distinguishability from fluorescent display colors.

In another example, the processing of block 200 converts the format of the image stream of image signal 27 from an original, first color space (preferably an 8-bit depth for each primary color, using primaries as defined in the BT-709 recommendation) into a new, second, data format (typically a 10-bit or 12-bit depth for each primary color, using primaries as defined in the BT-2020 recommendation) having a larger color space than the first color space, while preserving color space content of the first image stream. That is, the colors in the first image stream are kept the same despite a reformatting to a larger color space expressed with more bit depth and different primary colors.

In yet another example, color information on only two of the RGB channels may be expanded to a space with all three RGB channels, while FI images used on the third of the incoming RGB channels are processed differently. Circuit block 200 may access local lookup table (LUT) memory 208, or a LUT in system memory 56. Format conversion may be conducted with a LUT or directly calculated with a matrix multiplication of the RGB values in the first color stream. The processing of circuitry 200 will be further described below.

Similarly, for a fluorescence imaging modality, fluoresced data signal 29, circuitry may be provided optional processing steps, and then circuitry transforms the FI signal to an appropriate color range for display at circuit block 201. In other versions, FI image data may be carried in from the RGB channels of signal 27, and separated from the WL processing blocks, then fed to block 201. Block 201, which is an integer number Kth step of processing signal 29, formats the image stream represented by fluoresced data signal 29 to the second data format and transforms the image stream to a desired color range, as further described below.

The transformation of image data from signal 29 as well as the characteristics and design considerations for the various color spaces involved will be further described below, but may also involve local memory accessing a LUT (not shown) for each conversion. Further processing steps may be performed by additional circuit blocks following blocks 200 and 201, and preceding the imaging combining process performed at block 204. The further processing steps are preferably independent and may vary depending on the type of FI imaging employed, the application, user settings, and various other design considerations.

In this version, blocks 202 and 203 operate to, when the image processing circuitry is placed in the flashing mode, cause temporal modulation of pixel intensity of the FI signal depicting the fluoresced light component. Circuit block 202 is configured to supply a scaling signal that is used to oscillate or flash the FI imagery.

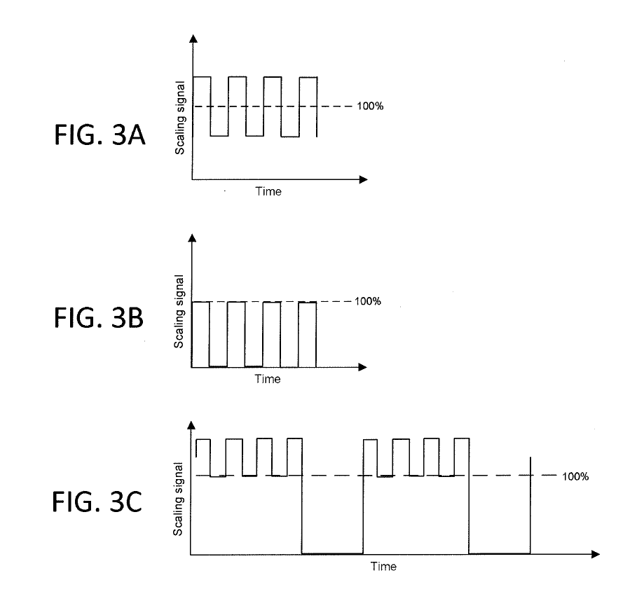

This signal is produced under control of oscillation control inputs, which are based on user settings and system configuration to control the FI image display. FIGS. 3A-C show example scaling signals that may be applied to scale the FI image data from block 201.

In the depicted circuitry of FIG. 2, a multiplication block 203 is used to scale luminance values of the FI images, however many other suitable methods may be used to alter the brightness of the FI imagery to obtain the desired flashing effect, as a suitable transform is available for every image data format to adjust the perceived brightness or luminance of the image. Similar adjustments may be applied to color or hue.

As shown in FIG. 3A, a scaling signal may be applied to each pixel of the FI image that varies the luminance of the FI image over time to oscillate it higher and lower than the original value, designated at 100%. The signal of FIG. 3A is multiplied with the FI images luminance values at block 203. For example, the values may be oscillated over time by multiplying each by 1.5, then by 0.5, then by 1.5. FIG. 3B shows a similar scheme except the luminance values are oscillated between zero and 100% (multiplication by 1). A value greater than 1 may also be used to oscillate with zero or near zero values.

FIG. 3C shows a scaling signal that may be applied at block 203 to both oscillate and pulse the brightness of the FI image. As seen, the scaling signal pulses the brightness or luminance during the ON cycle of the signal, and then oscillates to an OFF cycle with reduced or zeroed brightness. Many other suitable scaling signals may be used to oscillate or vary the FI image brightness. The rate or period of oscillation may be varied by is typically between 2 and 10 oscillations per second. A rate of 3 flashes per second has been found to be effective at attracting visual attention, for example. Further features may be provided in some embodiments to vary the oscillation rate in different portions of the FI image, as further described below.

Image processing circuitry 30 may provide one temporal modulation implementation by toggling coefficients of a color correction matrix ON/OFF and/or applying a scaling signal to said coefficients in a matter similar as described above, thereby causing the FI image stream (or upstream or downstream equivalent depicted in the composite image stream) to flash when displayed.

Referring again to FIG. 2, when the device is operated to produce fluorescence imaging, that is the FI modality is active, a flashing mode may be set on or off. When the flashing mode is set to on (which may be accomplished by a variety of controls in different versions), a scaling signal is applied at block 203, but is not applied when the flashing mode is set to off. Block 204 produces a composite image stream depicting the reflected light components and the fluoresced light component, as detected by the image sensor assembly. While this versions shows separate data paths for WL and FI data, in other versions, for the FI modality, the image forming circuitry is may be configured to receive the at least three FI output signals and produce a composite image stream depicting the reflected light components and the fluoresced light component detected by the image sensor assembly. Further, while this version shows the temporal modulation or oscillation applied at block 203, other versions may implement this feature in other ways (e.g., upstream or downstream of block 203), such as with a time-varying filter applied to a composite image.

After the image combination at block 204, further image processing steps may be performed on the combined image data 206. Data bus 52 next transports the combined image data 206 to the system controller, and may transfers other information both ways between the system controller 50 and each of the image processing blocks. The transported information typically includes image processing step parameters and user-selected option indicators.

In the illustrated embodiment, image processing circuitry 30 manipulates the digital image data according to processes that are either programmed into the circuit (in the case of programmable logic devices) or loaded into the circuit program memory as programming instructions (in the case of processors and controllers such as a graphics processing unit (GPU)).

The digital image data manipulation includes, but is not limited to, image processing steps such as color filter array demosaicing, noise reduction, color correction, image dewarping, and gamma correction. The image processing may further include frame syncing in designs where the frame rate of signal 29 is lower than that of signal 27. For example, if signal 27 includes 30 frames-per-second color images, but signal 29 has a longer sensor integration time and only contains 5 or 10 frames-per-second of fluoresced light data, image processing circuitry may need to hold, repeat, or interpolate frames between blocks 201 and 204 in order that the image combining process performed by block 204 is properly synced. In this version the digital image data manipulation performed by image processing circuitry 30 also includes and calculating control signals from each signal 27 and 29 such as exposure levels required by exposure controller 40 to adjust the imaging device for proper light levels in the detected light.

The various depicted circuitry blocks inside image processing circuitry 30 are preferably FPGA logic blocks inside a single FPGA device, which includes an on-chip controller and memory. However, this is not limiting and processors, ASICs, GPUs, and other suitable circuits may be used to implement the depicted circuitry blocks.

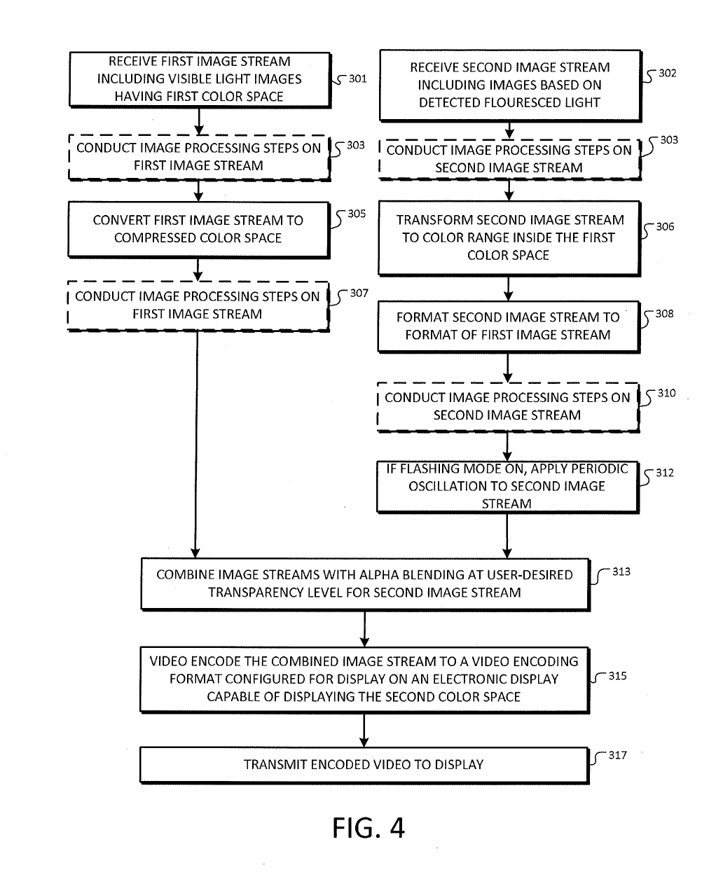

FIG. 4 is a flowchart of a combined color and fluoresced imaging process according to some embodiments, which may be employed with the example hardware and functional designs of FIGS. 1-2, or may be employed with other hardware designated with a similar purpose, such as software program code executed by a GPU or other image processor. Generally, the process gives a method of operating a fluorescence imaging scope system with WL and FI modalities.

Process block 301 includes receiving a first image stream, such as that in data signal 27 (FIG. 2), produced from detected visible light and formatted in a first bit depth expressing a first color space of visible light. In the presently preferred embodiment, the color space is defined an 8-bit or 10-bit depth for each primary color, using R, G, and B primaries as defined in the BT-709 recommendation. Other versions may use other bit depths, such as, for example, older sensor output formats that used 6 bits per color channel, or 7 or 9 bits, and other primary color definitions to define the first color space. The first color space is preferably defined by at least three primaries.

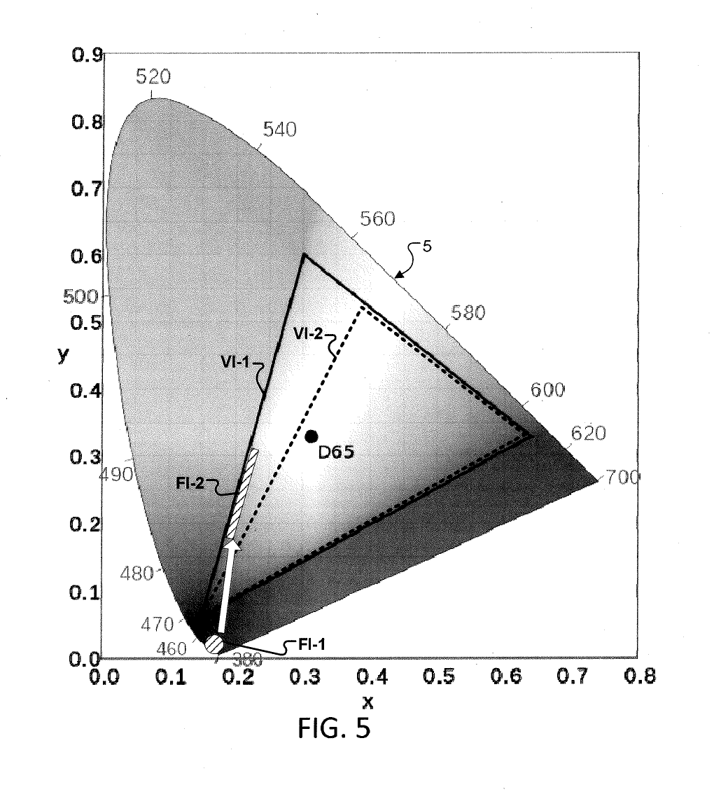

FIG. 5 shows an example color space diagram according to an embodiment in which the BT-709 8-bit per channel RGB color space is employed for the first image stream. The diagram is a CIE 1931 color space chromaticity diagram, with y parameter expressing a measure of the color's brightness or luminance, x expressing a measure of the human eye's response to colors, and the color's chromaticity being expressed as a function of the two parameters x and y. The CIE visible light diagram shows the area of colors visible to the human eye by the curved boundary, marked with arrow 5, which denotes monochromatic light, with wavelengths shown in nanometers. The standard's white point, D65, is shown in the diagram. Drawn onto the chromaticity diagram is a solid triangle marked VI-1, which shows the color space of the first image stream, which is the same color space defined by the bit depth and primaries of the first image stream.

Referring to FIG. 5 and also FIG. 4, to prepare the first image stream received at block 301 for combination with FI imagery and display, the process at block 303 conducts one or more image processing steps, with block 303 shown in dotted lines to indicate it is optional in some embodiments. Next at block 305, the process may optionally transform the images in the first image stream to compress the color space, thus allowing FI images to be combined with the first image stream without any color overlap, improving the ability to visually distinguish the FI images. The same bit format may be kept for the compressed color image stream. Recognizing that natural colors occurring in the first image stream (visible light imagery) do not often overlap all areas available in the color space, some versions may not alter the image stream at block 305. The altered first image stream color space is depicted in FIG. 5, with the compressed color range shown by the dotted triangle VI-2. It is noted that some versions may provide the depicted color space compression only when a fluorescent image is in flashing mode flashing on (block 312), and revert back to the full color range of the first image stream when the flashing mode oscillates to off or low intensity, providing an effect of slightly greying or dulling the visible image when the FI image portion is flashed on.

Other versions may perform the color compression of block 305 only when FI images are shown but are not in flashing mode, and maintain the full color space of the first image stream when FI images are in flashing mode for all stages of the flashing oscillation. The process may conduct further image processing steps at block 307.

Referring still to FIG. 4, in parallel to processing of the visible light images starting at block 301, the right-hand branch of the flowchart shows the FI images being processed. At block 302, the process receives a second (FI) image stream produced from detected first fluoresced light. A third image stream may also be processed similarly to the second image stream in some versions. Optional image processing steps are conducted at block 304 before the transformation.

At block 306, the process transforms the second image stream to a portion of the second color space outside compressed color space produced by block 305. If no compressed color space is used for the white-light images at block 305, block 306 preferably transforms the second image stream to a desired color or color range that has been chosen to be highly visible when overlaid with visible light images expected to be viewed with the scope.

One example of the transformation is depicted on the diagram of FIG. 5, where a depicted area showing fluoresced light spectrum is shown at signal FI-1, the light being detected and stored typically as intensity values interpreted as a grayscale image stream or FI image, even though all or some of the detected light in many embodiments is not actually visible light. The FI image stream containing signal FI-1 is shown being transformed as depicted by the arrow to a portion FI-2, which is inside the available display color space of VI-1, but outside the compressed color space VI-2.

For ICG FI applications, the fluorescence excitation light wavelength may be around 765 nm and FI-1 (e.g., the sensor-detected emission wavelength) may lie in the near-infrared spectrum (e.g., around 840 nm).

This allows the image of the fluorescent data to be displayed combined or overlaid with the visible color image. Transforming the second image stream may be done by accessing a lookup table containing a set of input pixel values for pre-transformed second images and an associated set of output pixel values for transformed second images. Transforming the second image stream may also be done by a transform algorithm executed by a processor or digital logic. Such an algorithm may include intensity to color transformation and intensity scaling as discussed above. Transforming the second image stream may also be done based on user configurable values for transparency, brightness, color, color range beginning, and color range end, for example. Transforming from second image stream may also include adding a transparency level to the second image stream, where combining the converted first image stream and the transformed second image stream (done at block 313) further comprises alpha blending in which the transparency level is used as the alpha level for the second image stream.

Next at block 308, the process formats the transformed image stream to the second data format, the same format used for the first stream. This process block may occur simultaneously with or before block 306. Further image processing may be conducted at block 310 before the image streams are combined.

At block 312, the process determines whether the image processing circuitry is placed in a flashing mode, and if so applies a temporal modulation of pixel intensity values of the FI image stream. This modulation is preferably a periodic oscillation, such as that described in the example scaling signals of FIGS. 3A-C. Preferably the oscillation is applied to the luminance intensity or other value controlling perceived brightness of the second image stream, but other oscillations may be applied either alone or in any combination. For example, the color may be altered along with the brightness.

Next, the image combining occurs at block 313, which combines the converted first image stream and the transformed second image stream into a combined image stream. The combination may be done by overlaying or alpha blending the images, or other suitable means of image combining. In a preferred version, the block 313 includes adding a transparency level to the second image stream, which may be set by the user through the user interface. Then combining the converted first image stream and the transformed second image stream is done by alpha blending in which the transparency level is used as the alpha level for the transformed second image stream.

Next at block 315, the process video encodes the combined image stream to a video encoding format configured for display on an electronic display capable of displaying the entire color space employed. Process block 317 then transmits this encoded signal for display on such a display.

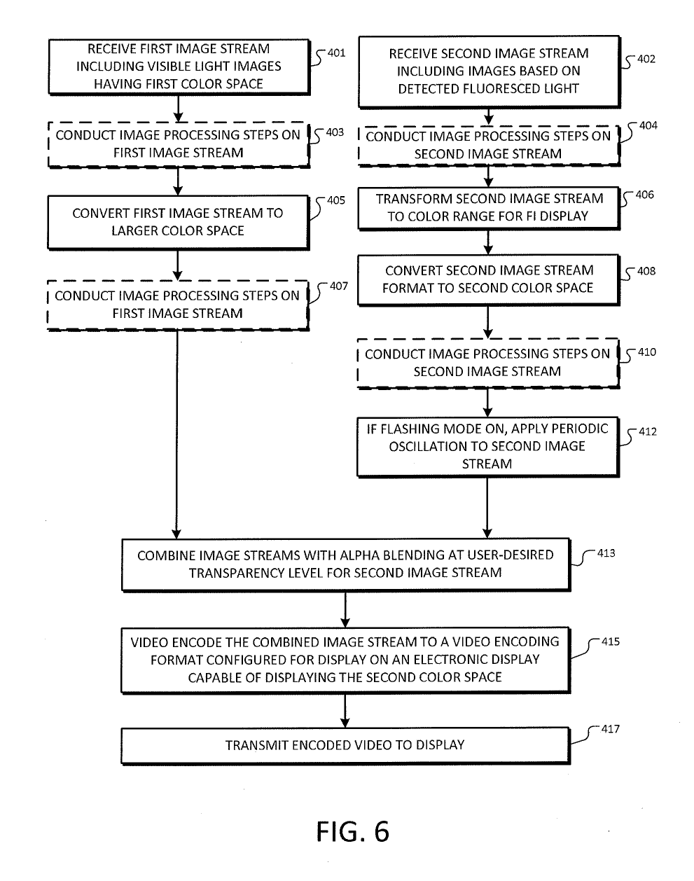

FIG. 6 is a flowchart of another fluorescent image and visible image display process according to other embodiments. This process has the same general steps as the process of FIG. 4, with the goal of emphasizing FI imagery when combined with visible light imagery. However, this version uses an enlarged color space to accommodate more colors with which to display the FI imaging. The difference is seen first at block 405, where the process converts the format of the first image stream to a larger color space. In some versions, this may involve mapping visible light images carried in two primary channels of an RGB color format onto the larger three-channel color space. This may involve color conversion or other processing to improve the color accuracy.

In other versions, block 405 may involve transforming from an original, first color space into a new, second, data format (typically a 10-bit or 12-bit depth for each primary color, using primaries as defined in the BT-2020 recommendation) having a larger color space larger than the first color space, while preserving color space content of the first image stream.

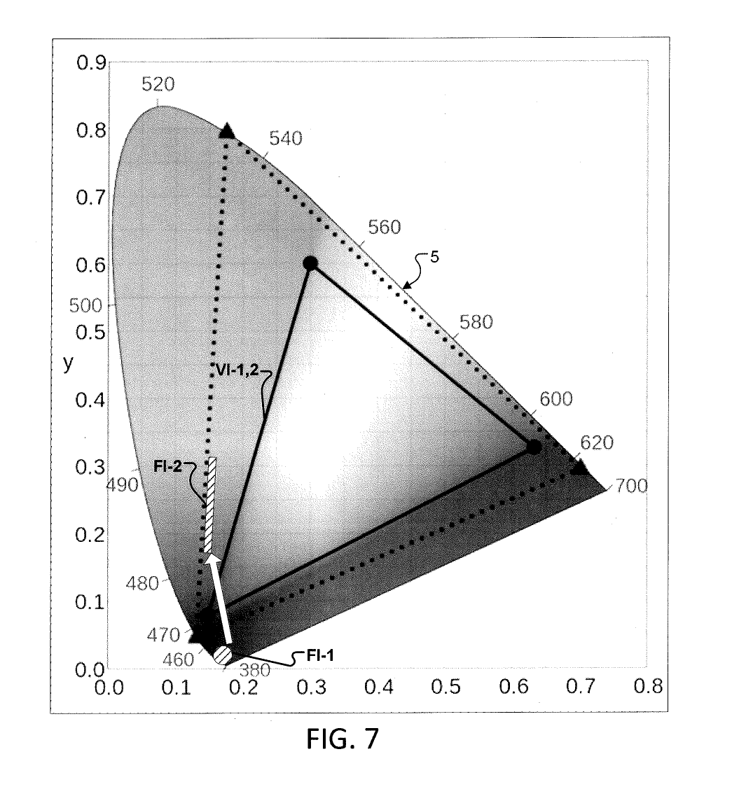

This is depicted in FIG. 7, with the second color space shown by the dotted triangle depicting the 10-bit color space using primaries as defined in the BT-2020 recommendation. While the first image stream data format is changed to the larger triangle, but the color space is not expanded so the colors in the first image stream remain unchanged at the original, smaller color space labeled VI-1,2. The format conversion at block 405 may be conducted with a lookup table (LUT) or directly calculated with a matrix multiplication of the RGB values in the first color stream. The second colors space is preferably defined by at least three or four primaries, however in some versions the visible light images are provided at block 401 on only two primaries of a three-channel system, and are converted to the larger, three-primary color space at block 405. In such versions, the third primary channel carries the FI image data received at block 402. The preferred version uses a three-primary color space as the second, larger, color space.

In parallel to processing of the visible light images starting at block 401, the right-hand branch of the flowchart shows the fluoresced light-based images being processed. At block 402, the process receives a second image stream produced from detected first fluoresced light. A third image stream may also be processed similarly to the second image stream. Optional image processing steps are conducted at block 404 before the transformation.

In versions where RGB channels are used to carry the FI data in the FI modality, generally the image sensor assembly is configured to produce at least three FI output signals for the FI modality, one or more (preferably two) of the at least three FI output signals including WL image signals, and preferably one channel carries the FI image data. If the FI data is already in a suitable color range and color space, no transformation is needed at blocks 406 and 408.

As shown at block 412, the image processing circuitry is configured to, when the image processing circuitry is placed in the flashing mode, cause temporal modulation of pixel intensity values of the composite image stream that represent the at least one FI output signal depicting the fluoresced light component. This may be done, for example, by alternating periodically between an off state that suppresses, of the three channels, the one or more channels providing the at least one FI output signal, and an on state that does not suppress the one or more channels.

At block 413, the image forming circuitry is configured to receive the at least three FI output signals and produce a composite image stream depicting the reflected light components and the fluoresced light component detected by the image sensor assembly.

For versions of the process in which color space transformation is employed, at block 406, the process transforms the second image stream to a portion of the second color space outside the first color space. One example of this transformation is depicted on the diagram of FIG. 7, where a depicted area showing fluoresced light spectrum is shown at signal FI-1, the light being detected and stored typically as intensity values interpreted as a grayscale image stream or FI image, even though all or some of the detected light in many embodiments is not actually visible light. The FI image stream containing signal FI-1 is shown being transformed as depicted by the arrow to a portion FI-2 of the second color space, which is outside the first color space. This allows the image of the fluorescent data to be displayed combined or overlaid with the visible color image, without requiring a change to the color space content of the color image itself

Transforming the second image stream may be done by accessing a lookup table containing a set of input pixel values for pre-transformed second images and an associated set of output pixel values for transformed second images. Transforming the second image stream may also be done by a transform algorithm executed by a processor or digital logic. Such an algorithm may include intensity to color transformation and intensity scaling as discussed above. Transforming the second image stream may also be done based on user configurable values for transparency, brightness, color, color range beginning, and color range end, for example. Transforming from second image stream may also include adding a transparency level to the second image stream, where combining the converted first image stream and the transformed second image stream further comprises alpha blending in which the transparency level is used as the alpha level for the second image stream.

Next at block 408, the process formats the transformed image stream to the second data format, the same format used for the first stream. This process block may occur simultaneously with or before block 406. Further image processing may be conducted after block 410 before the image streams are combined. Next at block 412, similarly to the flowchart of FIG. 4, the process determines whether the image processing circuitry is placed in a flashing mode, and if so applies a periodic oscillation to the second image stream, such as that described in the example scaling signals of FIGS. 3A-C.

The combining occurs at block 413, which combining the converted first image stream and the transformed second image stream into a combined image stream. The combination may be done by overlaying or alpha blending the images, or other suitable means of image combining. In a preferred version, the block 414 includes adding a transparency level to the second image stream, which may be set by the user through the user interface. Then combining the converted first image stream and the transformed second image stream is done by alpha blending in which the transparency level is used as the alpha level for the transformed second image stream.

Next at block 415, the process video encodes the combined image stream to a video encoding format configured for display on an electronic display capable of displaying the second color space. Preferably such display is a 4K or other UHD monitor or television configured to display the 10-bit or 12-bit color space discussed above as defined by the BT-2020 ITU recommendation. Block 417 then transmits this encoded signal for display on such a display.

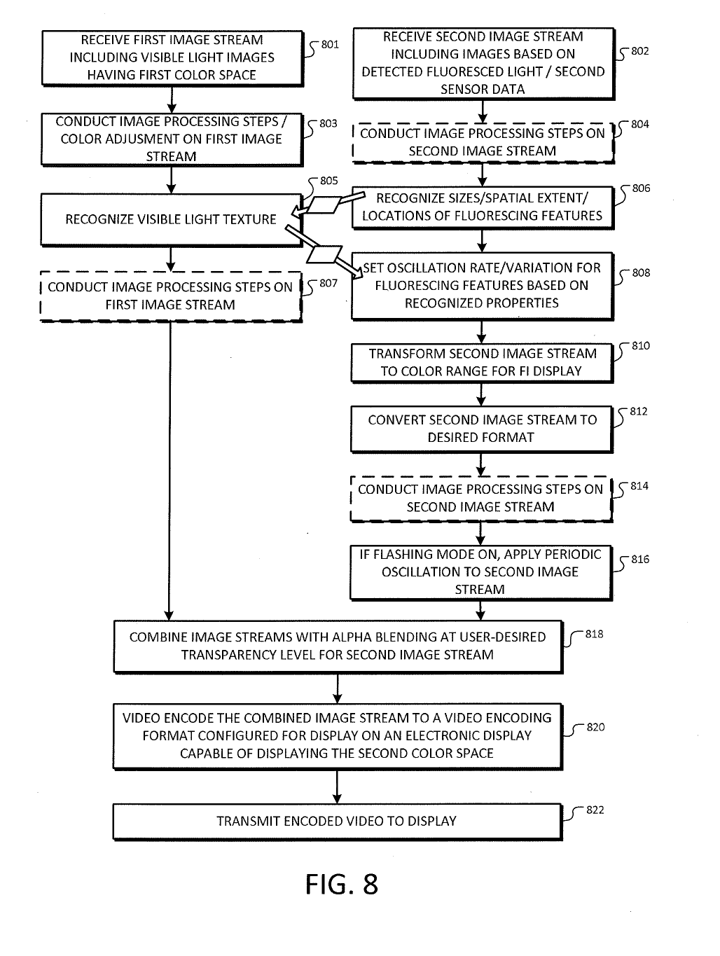

FIG. 8 is a flowchart of another example process including image processing to recognize elements for emphasis in the display. In this version, several image processing techniques are employed to control the flashing or oscillating of the fluorescent display techniques which may be combined with many other embodiments of the invention including the examples of FIG. 4 and FIG. 6.

The depicted process generally proceeds similarly to the FIG. 4, including processing two image streams and then combining them. The order of the techniques is not limiting unless the output of a particular step is required as the input of another. Preferably the image processing steps herein are performed in parallel according to the circuitry described with regard to FIG. 2, or other suitable image processing circuitry. The process receives a first image stream of visible light images at block 801 and conducts any required image processing steps and color adjustment such as, for example color space compression or color space format adjustment, at block 803.

In parallel, a second image stream based on detected fluoresced light is receive at block 802 with optional image processing steps performed at block 804. Next at block 806, image processing is performed to identify properties of fluorescing features. Many suitable properties may be recognized or calculated at this block, including the size of fluoresced features, their spatial extent (based on the area or area and angle of surfaces in the image), and their locations and frequency of appearance within areas of the image.

For example, this block may recognize an area with a concentration of many small fluorescing features. The results of these processing steps may also be used in combination with processing of the visible light images as shown by the data passing to block 805 in the FIG. 8. In particular, block 806 may pass a set of identified fluorescing feature parameters to block 805 including the sizes and locations of the features. Block 806 may also pass information identifying areas with a relatively high density of small fluorescing features as compared to other areas in the FI images. Block 805 determines the surface texture in these areas using the visible light images to recognize texture properties such as a roughness, texture energy, or texture complexity, for example. Such recognized properties are passed to the processing stream for the FI imaging at block 808. This block sets the oscillation of flashing rate for individual features or image zones (areas) of the image based at least one of the properties calculated at block 806 and at least one of the received properties from block 805. Some features may have their oscillation rate set based on only the properties from block 806.

Block 808 may also set other parameters such as the range of intensity variations in the flashing mode, and the level around which variations are displayed (for example, selecting a scaling signal from among various types such as those in FIGS. 3A-C). For example, in one version smaller fluorescing areas, which may in a particular medical exam represent small lesions, may be made to flash faster than other larger regions, or to flash more intensely (with a higher intensity variation of oscillations), or both. Next at blocks 810-814 the process performs steps already discussed to prepare the second image stream for combination. The flashing or oscillation is applied at block 816 (which may be done at any point after block 808 where the flashing parameters are set). Finally the image streams are combined at block 818, encoded at block 820, and transmitted for display at block 822, similarly to the other example processes discussed herein.

The techniques discussed above may be implemented in a variety of hardware designs, and signal processing software designs. The design should be conducted considering the need for real-time image display, that is, to minimize lag on the display as the scope is moved by medical personnel. The parallel hardware design of FIG. 2 is therefore advantageous because it adds little processing time to the slower parts of the system, that is the image processing steps conducted on the full color visible image or the combined image.

It can also be understood, after appreciating this disclosure, that the techniques herein may be employed in other fields that include combining fluorescent imagery with visible light imagery, such as microscopy.

As used herein the terms “comprising,” “including,” “carrying,” “having” “containing,” “involving,” and the like are to be understood to be open-ended, that is, to mean including but not limited to. Any use of ordinal terms such as “first,” “second,” “third,” etc., in the claims to modify a claim element does not by itself connote any priority, precedence, or order of one claim element over another, or the temporal order in which acts of a method are performed. Rather, unless specifically stated otherwise, such ordinal terms are used merely as labels to distinguish one claim element having a certain name from another element having a same name (but for use of the ordinal term).

The foregoing has outlined rather broadly the features and technical advantages of the invention in order that the detailed description of the invention that follows may be better understood. It should be appreciated by those skilled in the art that the conception and specific embodiments disclosed may be readily utilized as a basis for modifying or designing other structures for carrying out the same purposes of the invention. It should also be realized by those skilled in the art that such equivalent constructions do not depart from the scope of the invention as set forth in the appended claims.

Although the invention and its advantages have been described in detail, it should be understood that various changes, substitutions and alterations can be made herein without departing from the scope of the invention as defined by the appended claims. The combinations of features described herein should not be interpreted to be limiting, and the features herein may be used in any working combination or sub-combination according to the invention. This description should therefore be interpreted as providing written support, under U.S. patent law and any relevant foreign patent laws, for any working combination or some sub-combination of the features herein.

Moreover, the scope of the present application is not intended to be limited to the particular embodiments of the process, machine, manufacture, composition of matter, means, methods and steps described in the specification. As one of ordinary skill in the art will readily appreciate from the disclosure of the invention, processes, machines, manufacture, compositions of matter, means, methods, or steps, presently existing or later to be developed that perform substantially the same function or achieve substantially the same result as the corresponding embodiments described herein may be utilized according to the invention. Accordingly, the appended claims are intended to include within their scope such processes, machines, manufacture, compositions of matter, means, methods, or steps.

1. A fluorescence imaging scope system capable of white-light (WL) and fluorescence imaging (FI) modalities, comprising:
an optical assembly configured to direct light received from a subject scene toward an image sensor assembly;
an image sensor assembly with at least three channels and including at least one image sensor, the image sensor assembly configured to:
detect reflected light components and a fluoresced light component of the light, and
produce at least three WL output signals for a WL modality and at least one FI output signal depicting the fluoresced light component for an FI modality;
image forming circuitry configured to receive the at least three WL output signals and produce a WL image stream, and receive the at least one FI output signal and produce an FI image stream; and
image processing circuitry configured to, when the image processing circuitry is in a flashing mode, cause temporal modulation of pixel intensity values of the FI image stream through signal processing.

2. The fluorescence imaging scope system of claim 1, wherein the image sensor assembly is configured to produce at least three FI output signals for the FI modality, one or more of the at least three FI output signals depicting the reflected light components,
the image forming circuitry is configured to receive the at least three FI output signals and produce a composite image stream depicting the reflected light components and the fluoresced light component detected by the image sensor assembly, and
the image processing circuitry configured to, when the image processing circuitry is in the flashing mode, cause temporal modulation of pixel intensity values of the composite image stream that represent the at least one FI output signal depicting the fluoresced light component through signal processing.

3. The fluorescence imaging scope system of claim 1, wherein the image processing circuitry is configured to, when the image processing circuitry is in the flashing mode, temporally modulate the at least one FI output signal or the FI image stream.

4. The fluorescence imaging scope system of claim 1, wherein the image processing circuitry is configured to, when the image processing circuitry is in the flashing mode, to alternate periodically between an off state that suppresses, of the at least three channels, the one or more channels providing the at least one FI output signal, and an on state that does not suppress the one or more channels.

5. The fluorescence imaging scope system of claim 1, wherein the at least three channels include a red channel, a green channel, and a blue channel.

6. The fluorescence imaging scope system of claim 1, in which the image processing circuitry is further configured to compress a color space of the WL image stream to a color space not containing a fluorescence display color range for the FI image stream.

7. The fluorescence imaging scope system of claim 1, in which the WL image stream has a first color space, and the image processing circuitry is further configured to convert a format of the WL image stream into a second data format having a second color space larger than the first color space, while preserving color space content of the WL image stream, and to format the FI image stream to a color format inside the second color space and outside the first color space.

8. The fluorescence imaging scope system of claim 1, in which the image processing circuitry is further configured to receive an external user interface control input signal controlling the flashing mode.

9. The fluorescence imaging scope system of claim 1, in which the image processing circuitry is further configured to receive a flashing rate input for adjusting a flashing rate of the flashing mode.

10. The fluorescence imaging scope system of claim 1, in which the image processing circuitry is further configured to assign multiple different flashing rates to respective multiple different areas of the FI image stream based on digital image processing values calculated from the respective areas.

11. The fluorescence imaging scope system of claim 1 in which the image processing circuitry further includes first processing circuitry for processing the WL image stream and second processing circuitry operating in parallel with the first processing circuitry for processing the FI image stream, the first and second processing circuitry both connected to image combining circuitry, and further in which the first and second processing circuitry comprise independent parallel circuits in a field programmable gate array (FPGA).

12. A camera control module (CCM) for commutatively coupling with a fluorescent and visible light medical scope device, the CCM comprising:
a scope connection port configured to receive at least one output signal from a scope device, the at least one output signal including detected reflected light components for a white-light (WL) modality and detected fluoresced light components for a fluorescence imaging (FI) modality from the scope device;
image forming circuitry configured to receive the at least one output signal and produce a WL image stream and a FI image stream; and
image processing circuitry configured to, when the image processing circuitry is in a flashing mode, cause temporal modulation of pixel intensity values of the FI image stream through signal processing.

13. The camera control module of claim 12, wherein the CCM is configured to receive at least three FI output signals for the FI modality, one or more of the at least three FI output signals depicting the reflected light components,
the image forming circuitry is configured to receive the at least three FI output signals and produce a composite image stream depicting the reflected light components and the fluoresced light component, and
the image processing circuitry configured to, when the image processing circuitry is in the flashing mode, cause temporal modulation of pixel intensity values of the composite image stream that represent the fluoresced light component through signal processing.

14. The camera control module of claim 13, wherein the at least three FI output signals include a red channel, a green channel, and a blue channel and the image processing circuitry is configured to, when the image processing circuitry is in the flashing mode, temporally modulate the composite image stream or the one or more of the at least three FI output signals depicting the reflected light components.

15. The camera control module of claim 12, in which the image processing circuitry is further configured to compress a color space of the WL image stream to a color space not containing a fluorescence display color range for the FI image stream.

16. The camera control module of claim 12, in which the WL image stream has a first color space, and the image processing circuitry is further configured to convert a format of the WL image stream into a second data format having a second color space larger than the first color space, while preserving color space content of the WL image stream, and to format the FI image stream to a color format inside the second color space and outside the first color space.

17. The camera control module of claim 12, wherein the image processing circuitry is further configured to assign multiple different flashing rates to respective multiple different areas of the composite image stream based on digital image processing values calculated from the respective areas.

18. The camera control module of claim 12, in which the image processing circuitry further includes first processing circuitry for processing the WL image stream and second processing circuitry operating in parallel with the first processing circuitry for processing the FI image stream, the first and second processing circuitry both connected to image combining circuitry, and further in which the first and second processing circuitry comprise independent parallel circuits in a field programmable gate array (FPGA).

19. One or more tangible nontransitory computer readable media storing program code executable by a digital processing system to perform the following:
receive at least three signals depicting reflected light components for a WL modality and produce a WL image stream therefrom, and receive FI data depicting a fluoresced light component for an FI modality and produce an FI image stream therefrom; and
when the digital processing system is placed in a flashing mode, temporally modulate one of the FI data and the FI image stream, thereby causing temporal modulation of the pixel intensity values of the FI image stream through signal processing.

20. The computer readable media of claim 19, wherein the program code is further executable by the digital processing system to perform the following:
produce a composite image stream depicting the reflected light components and the fluoresced light component, and
when the digital processing system is in the flashing mode, temporally modulate the fluoresced light component through signal processing.

21. The computer readable media of claim 19, wherein the program code is further executable by the digital processing system to perform the following:
receive at least three channels for the FI modality, with data for producing the WL image stream carried by one or more of the three channels, to produce a composite image stream depicting the reflected light components and the fluoresced light component detected by the image sensor assembly, and when the digital processing system is in the flashing mode, to alternate periodically between an off state that suppresses, of the three channels, the one or more channels providing the FI data, and an on state that does not suppress the one or more channels.

22. The computer readable media of claim 19, wherein the program code is further executable by the digital processing system to assign multiple different flashing rates to respective multiple different areas of the FI image stream based on digital image processing values calculated from the respective areas.
